(19) United States
(12) Patent Application Publication
(10) Pub. No.: US 2024/0408378 A1
(43) Pub. Date: Dec. 12, 2024

(54) DELIVERY SYSTEMS AND METHODS FOR INFLOW / OUTFLOW CANNULAS

(71) Applicant: W. L. Gore & Associates, Inc., Newark, DE (US)

(72) Inventors: Scott M. Bryson, Newark, DE (US); Daniel Dominguez, Anthem, AZ (US); James L. Goepfrich, Flagstaff, AZ (US); Joshua C. Haarer, Flagstaff, AZ (US); Edward E. Shaw, Flagstaff, AZ (US)

(21) Appl. No.: 18/698,859

(22) PCT Filed: Oct. 6, 2022
(86) PCT No.: PCT/US2022/045886

§ 371 Date: Apr. 5, 2024

Related U.S. Application Data
(60) Provisional application No. 63/253,410, filed on Oct. 7, 2021.

(51) Int. Cl. A61M 60/857; A61M 60/10; A61M 60/20; A61M 60/40; A61M 60/861

(52) U.S. Cl. CPC A61M 60/857; A61M 60/10; A61M 60/20; A61M 60/40; A61M 60/861; A61M 2205/0238

(57) ABSTRACT

Various aspects of the present disclosure are directed toward apparatuses, systems, and methods that may include an inflow or outflow cannula apparatus.

### CROSS-REFERENCE TO RELATED APPLICATION

This application is a national phase application of PCT Application No. PCT/US2022/045886, internationally filed on Oct. 6, 2022, which claims the benefit of Provisional Application No. 63/253,410, filed Oct. 7, 2021, which are incorporated herein by reference in their entireties for all purposes.

### FIELD

This disclosure relates generally to medical devices, and more specifically, to inflow or outflow cannulas that may include a tissue anchor.

### BACKGROUND

Cannulas may be able to create flow conduits within a patient. For example, cannulas may be used to create flow conduits to or from an organ such as the heart. U.S. Pat. No. 10,849,653 to Tao et al., describes a cannula supporting a percutaneous pump that includes a proximal section with a first flexural modulus and one or more distal sections with a flexural modulus that is different than the first flexural modulus. The material and its arrangement along the length of the cannula can be selected so as to influence bending properties. This can, for example, allow efficient positioning of the cannula in a desired location without displacing the guidewire. Though Tao provides an example of one use, cannulas may be used in other contexts, such as for interconnecting or bypassing portions of the vasculature (e.g., between one or more vessels).

### SUMMARY

According to one example (“Example 1”), a delivery system includes a cannula having an anchor structure configured from transition from a first, delivery configuration to a second, deployed configuration; a delivery sheath; and a delivery catheter including a pusher configured to advance the capsule relative to the cannula, the capsule being arranged at an end of the delivery catheter, the delivery catheter being configured to extend through the cannula and operable to advance the capsule through an opening, deploy a first portion of the anchor structure on a first side of the opening from the capsule, and deploy a second portion of the anchor structure against a second side of the opening from the capsule such that the anchor structure defines the second, deployed configuration, and optionally collapse the capsule to be withdrawn through the cannula from the second, deployed configuration to the first, delivery configuration after deployment of the anchor structure in response to force applied to the delivery catheter by a physician.

According to another example (“Example 2”), further to the delivery system of Example 1, the capsule is segmented to facilitate collapse and withdrawal through the cannula to the first, delivery configuration after deployment of the anchor structure.

According to another example (“Example 3”), further to the delivery system of Example 2, the capsule is discontinuous about a circumference of the capsule to facilitate collapse of the capsule in response to tension applied to the delivery catheter.

According to another example (“Example 4”), further to the delivery system of Example 3, the capsule includes a series of segments extending along a length and spaced about the circumference of the capsule.

According to another example (“Example 5”), further to the delivery system of Example 4, the capsule includes a tapered proximal end and a tapered distal end to facilitate collapse and withdrawal through the cannula after deployment of the anchor structure.

According to another example (“Example 6”), further to the delivery system of Example 5, wherein the tapered distal end of the capsule includes a continuous outer circumference and the tapered proximal end includes portions of the series of segments.

According to another example (“Example 7”), further to the delivery system of Example 6, the tapered proximal end is configured to facilitate collapse of the capsule within the cannula in response to tension applied to the delivery catheter to draw the capsule into the cannula.

According to another example (“Example 8”), further to the delivery system of Example 7, further comprising an elastic tube configured to constrain the capsule.

According to another example (“Example 9”), further to the delivery system of any one of Examples 1-8, the capsule includes a hydrophilic coating configured to facilitate visualization of the capsule and advancement of the capsule through the opening.

According to another example (“Example 10”), further to the delivery system of any one of Examples 1-9, further comprising a hemostasis plug.

According to one example (“Example 11”), a delivery system for a cannula including an anchor structure includes a hub configured to facilitate entry of the cannula into a patient at an entry point; a balloon catheter configured to extend through the hub and dilate a target location for the cannula and anchor structure; and a delivery sheath configured to extend between the entry point and the target location, the delivery sheath including: a distal end portion configured to constrain the anchor structure in a delivery configuration and deploy the anchor structure in response to a force applied to the delivery sheath to move the distal end portion relative to the cannula, the distal end portion optionally being operable to be peeled or torn from about the cannula.

According to another example (“Example 12”), further to the delivery system of Example 11, the hub is configured to facilitate splitting of the delivery sheath to remove the delivery sheath from about the cannula.

According to another example (“Example 13”), further to the delivery system of Examples 11 or 12, the balloon catheter is configured to hold the cannula at the target location while the delivery sheath is withdrawn to deploy the anchor structure.

According to another example (“Example 14”), further to the delivery system of Examples 11 to 13, the delivery sheath comprises expanded polytetrafluoroethylene (ePTFE).

According to another example (“Example 15”), further to the delivery system of Examples 11 to 14, the distal end portion includes a greater hoop strength than other portions of the delivery sheath.

According to one example (“Example 16”), a delivery system includes a cannula having an anchor structure arranged with the cannula; a delivery catheter including a balloon configured dilate a target location for the cannula and anchor structure; and a zipper constraint configured to constrain the anchor structure against the cannula in a delivery configuration and release to deploy the anchor structure in response to a force applied to a deployment line coupled to the zipper constraint by a user.

According to another example (“Example 17”), further to the delivery system of Example 16, the delivery catheter and the balloon are configured to provide column strength to the cannula for trackability while also serving as an inflatable lead to reduce trauma to vasculature tissue.

According to another example (“Example 18”), further to the delivery system of Example, the zipper constraint is configured constrain the anchor structure to the cannula to reduce a profile of the delivery system during delivery of the cannula to a target location.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of an example cannula and anchor structure, in accordance with various aspects of the present disclosure.

FIGS. 2A-B are illustrations of an example cannulas arranged within a patient's heart, in accordance with various aspects of the present disclosure.

FIG. 3 is an illustration of an example delivery system for a cannula, in accordance with various aspects of the present disclosure.

FIG. 4 is an illustration of the delivery system and cannula, as shown in FIG. 3, in a first configuration, in accordance with various aspects of the present disclosure.

FIG. 5 is an illustration of the delivery system and cannula, as shown in FIGS. 3-4, in a second configuration, in accordance with various aspects of the present disclosure.

FIG. 6 is an illustration of the delivery system and cannula, as shown in FIGS. 3-5, in a third configuration, in accordance with various aspects of the present disclosure.

FIG. 7 is an illustration of the delivery system and cannula, as shown in FIGS. 3-6, in a fourth configuration, in accordance with various aspects of the present disclosure.

FIG. 8 is an illustration of the delivery system and cannula, as shown in FIGS. 3-7, in a fifth configuration, in accordance with various aspects of the present disclosure.

FIG. 9 is an illustration of the delivery system and cannula, as shown in FIGS. 3-8, in a sixth configuration, in accordance with various aspects of the present disclosure.

FIGS. 10A-10C illustrate an example capsule for the delivery system, shown in FIGS. 8, in accordance with various aspects of the present disclosure.

FIG. 11 is an illustration of another example delivery system for a cannula, in accordance with various aspects of the present disclosure.

FIG. 12 is an illustration of the example delivery system, shown in FIG. 10, in a second configuration, in accordance with various aspects of the present disclosure.

FIG. 13 is an illustration of the example delivery system, shown in FIGS. 10-11, in a third configuration, in accordance with various aspects of the present disclosure.

FIG. 14 is an illustration of another example delivery system for a cannula, in accordance with various aspects of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed to apparatuses, systems, and methods that include a cannula. The cannula may be arranged within a patient and create a fluid passageway. The cannula may extend from an organ (e.g., vasculature, heart, gallbladder, liver, or lungs) and connect to a device, another organ, or bodily structure or exit the patient. In certain instances, the cannula may be a graft or stent graft that is arranged within the vasculature. The apparatuses, systems, and methods of the present disclosure may be used in improving or assisting the vascular and/or cardiac function of the heart, for example. The cannula, as discussed in further detail below, may include an anchor structure. The anchor structure may be arranged to anchor with a tissue between two compartments (e.g., two vessels, heart chambers, or other bodily structure) within a patient. The anchor structure may be arranged between vessels or between different portions of the same vessel, or on either side of a septum within a patient's heart, for example. The disclosed anchor structure or inlet portion of the cannula may be used with a pump device or system, such as a vascular pump or circulatory assist device for managing (e.g., increasing) blood flow in a patient.

The cannula may be delivered to a target location within the patient by a delivery system, as discussed in further detail below. The delivery system may be used to place the cannula and the anchor structure coupled or attached to an end of the cannula at the target location. In certain instances, the delivery system may be used to cross or open a space between compartments of the patient or within tissue of the patient to place the cannula and the anchor structure at the target location.

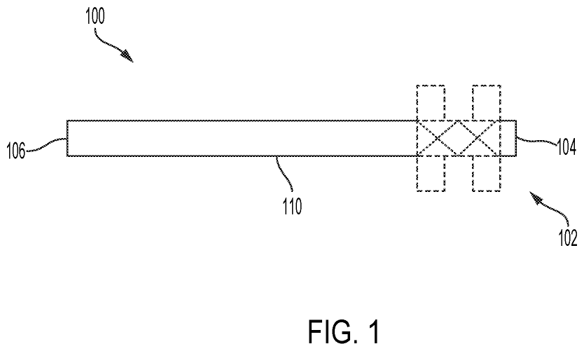

FIG. 1 is a schematic illustration of an example cannula 100 and anchor structure 102, in accordance with various aspects of the present disclosure. The anchor structure 102 may be used for an inflow or outflow cannula. Or, in different terms, the cannula may be configured as an inflow cannula that conveys fluid into a desired location (e.g., a pump or location in a body of a patient) or an outflow cannula that conveys fluid from a desired location (e.g., a pump location in a body of a patient). As shown, the cannula 100 has a first end 104 and a second end 106 opposite the first end 104. In certain instances, the anchor structure 102 may be arranged closer to the first end 104 of the cannula than 100 the second end 106 of the cannula 100. For example, the anchor structure 102 may be located at or near the first end 104 of the cannula 100.

The anchor structure 102 may include a single flange extending circumferentially outwardly from the cannula 100 or a plurality of flanges (e.g., a dual flange structure as illustrated generally in FIG. 1) extending circumferentially outwardly from the cannula 100 and/or a support structure arranged along a portion of the cannula 100 (e.g., on the outside of the cannula 100). The anchor structure 102 may be configured to transition from a first delivery configuration to a second deployed configuration. The first, delivery configuration may be a collapsed, delivery configuration and the second deployed configuration may be an expanded deployed configuration. Portions of the anchor structure 102 may be self-expanding, balloon expandable, or one or more portions of the anchor structure 102 may be self-expanding and one or more may be balloon expandable. The anchor structure 102 may facilitate against kinking or collapse of the end of the cannula 100 by reinforcing the cannula 100.

The cannula 100 may include a conduit 110 that is formed of biocompatible material, such as a graft material, and the anchor structure 102 may be formed of biocompatible material, such as that associated with stent elements and/or graft materials.

In certain instances, the conduit 110 may be relatively inextensible or may be configured to stretch to adjust in size, or may be configured to be stretched in response to forces acting on the conduit 110 and recover all or some portion of its length upon removal or reduction of such forces acting on the conduit 110. Suitable examples of graft materials may be found in U.S. Pat. No. 4,877,661 (“House, et al.”), although a variety of suitable materials are contemplated.

Any number of cannulas may be implemented, and the disclosure is not to be read in a limiting manner regarding the scope of the cannula. In one example, the cannula 100 is formed of a graft structure that may include reinforcing elements such as rings or a helical structure (e.g., helically wound wire or a tube with a helical cut extending around and longitudinally along the length of the cannula 100). In some embodiments, the cannula 100 may include a coating or material (e.g., a graft material such as, but not limited to ePTFE) for forming a fluid impermeable barrier for the cannula 100.

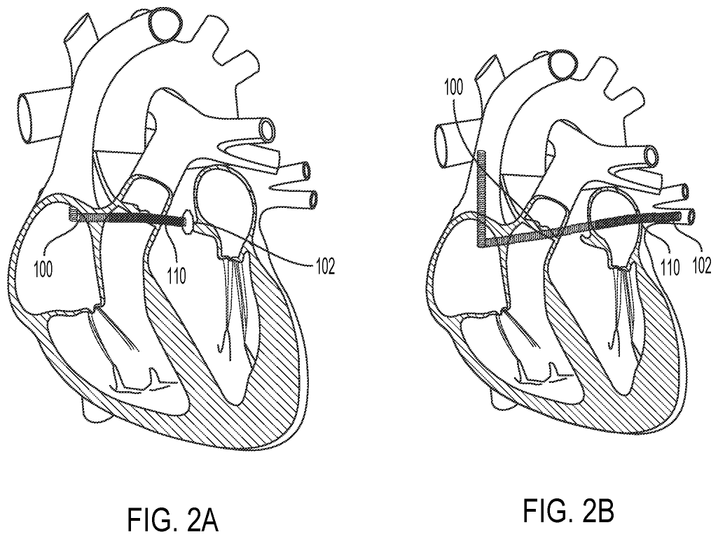

FIG. 2A is an illustration of an example of the cannula 100 shown arranged within a patient's heart, according to some embodiments. In the example shown, the cannula 100 may be used for an inflow or outflow cannula. As shown, the anchor structure 102 is arranged at or adjacent to an end of the conduit 110 of the cannula 100. The conduit 110 may be configured to provide a fluid passageway from or to a target location or between two target locations.

In certain instances, and as is shown, the anchor structure 102 is engaged with a septum of a patient's heart. In certain instances, the conduit 110 may be anchored within an opening in the body of the patient, such as a pulmonary vein of the patient. In other instances, the anchor structure 102 may be arranged with another organ, tissue structure, septum, tissue wall, opening, or within the vasculature of the patient.)

FIG. 2B shows another example positioning of the cannula 100 at a target location within the patient's heart. The cannula 100 may include a stent element, as described above with reference to FIG. 1 and may be anchored within the pulmonary vein. The cannula 100, when connected to a pump, may pull blood from either the pulmonary vein or a left atrium through the cannula 100 toward the pump.

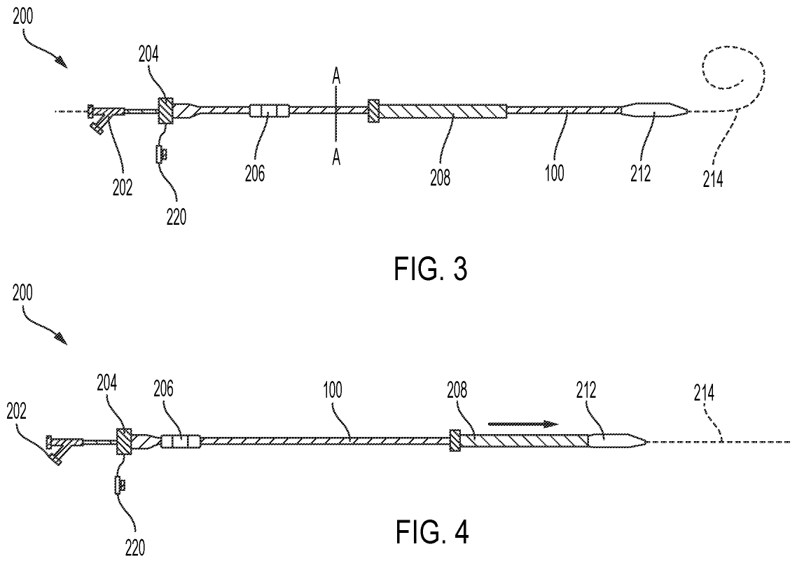

FIG. 3 is an illustration of an example delivery system 200 for a cannula 100, in accordance with various aspects of the present disclosure. The delivery system 200 may facilitate delivery of the cannula 100 to a target location within the patient. The delivery system 200 may be used to place the cannula 100 and the anchoring structure 102 (shown in FIGS. 6-9) coupled or attached to an end of the cannula 100 at the target location. In certain instances, the delivery system 200 may be used to cross or open a space between compartments of the patient or within tissue of the patient to place the cannula 100 and the anchor structure 102. The delivery system 200, as described in further detail below, may include a capsule 212 configured to facilitate crossing or opening a space between compartments of the patient or within tissue of the patient to place the cannula 100 and the anchor structure 102. In certain instances, the delivery system 200 also includes a hemostasis plug 204 that provides hemostasis between the cannula 100 and a delivery catheter 202. In certain instances, the hemostasis plug 204 may continue to provide hemostasis after the cannula 100 is arranged at the target location. The hemostasis plug 204 may be additionally configured with a flush port 220 to facilitate air management.

In certain instances, the delivery system 200 includes a delivery sheath 208, which is optionally configured as, and described as, a peel-away sheath 208 (shown in FIGS. 4-9). The delivery system 200 also includes the delivery catheter 202 that is configured to extend through the cannula 100, the capsule 212 arranged at an end of the delivery catheter 202, and a peel-away sheath 208 arranged about the cannula 100 in a first configuration (e.g., as shown in FIG. 3). The capsule 212 may receive the anchor structure 102 in the first, delivery configuration. In certain instances, a guidewire 214 may be used to direct the delivery system 200 to the target location. In certain instances, the guidewire 214 is introduced into a subclavian vein and directed toward the target location (e.g., left atrium). In certain configurations, the peel-away sheath 208 may alternatively be a straight introducer sheath, a coil or braid reinforced sheath, a shape-set sheath, a steerable sheath, or an expandable sheath.

As shown in FIG. 4, the cannula 100 and the anchor structure 102 (shown in FIGS. 6-9) may be extended toward a target location through the peel-away sheath 208. The anchor structure 102 may be arranged within the capsule 212 and the peel-away sheath 208 may be arranged adjacent the capsule 212 for initial introduction into the target location or into a target vessel. The cannula 100 and capsule 212 may be advanced to the target location (e.g., an organ such as the heart).

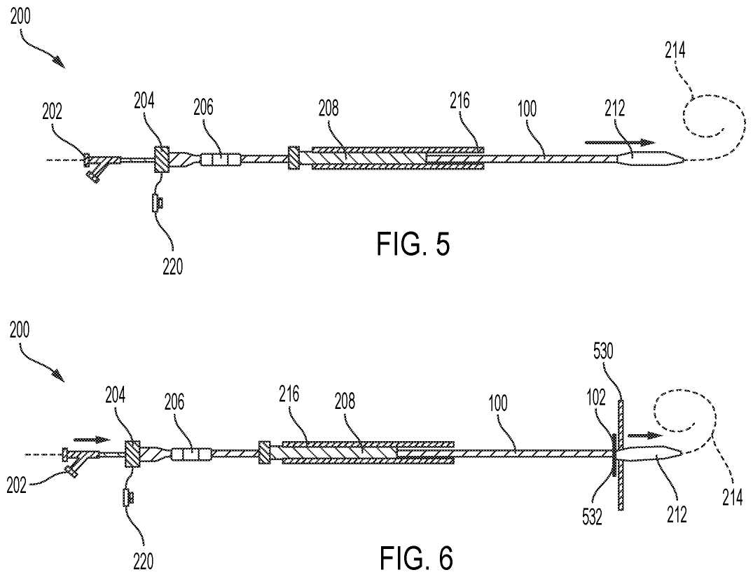

As shown in FIG. 5, the peel-away sheath 208 may be introduced, delivered, or otherwise positioned into a body of a patient, for example into a vasculature 216 of a patient. The vasculature 216 is represented schematically in FIG. 5 and may be a subclavian vein, for example.

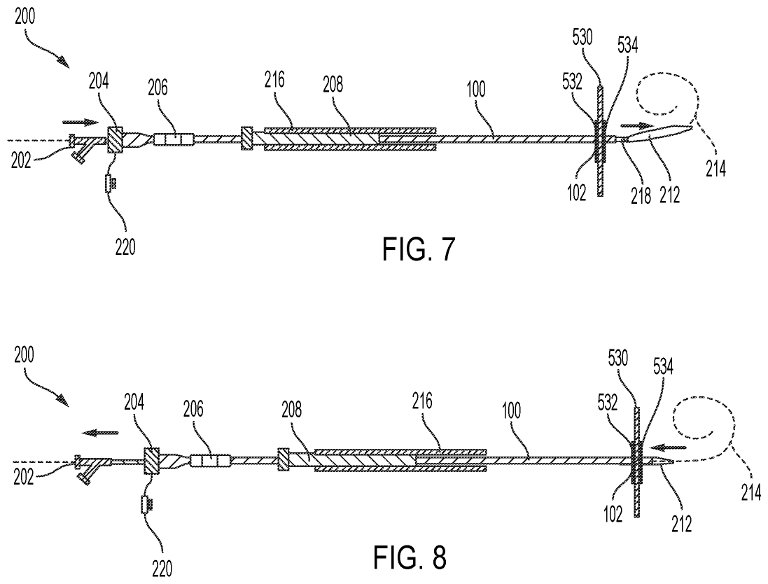

As shown in FIG. 6, the capsule 212 may be arranged across a target location 530 by advancing the delivery catheter 202. The target location 530 may be a tissue wall 530, which may include a heart wall, septum, a vessel wall, or other tissue wall, for example. After the capsule 212 is advanced past the end of the cannula 100 through an opening in the tissue wall 530 in response to advancing of the delivery catheter 202, a first portion 532 of the anchor structure 102 may be deployed on a first side of the tissue wall 530 in response to a force applied to the delivery catheter 202. For example, the delivery catheter 202 may include a pusher 218 (FIG. 7, also described as a pusher portion) coupled to the capsule 212. The delivery catheter 202 may be advanced to, in turn, advance the pusher 218, and thus the capsule 212, to release or free the anchor structure 102 from the capsule 212. This relative movement may include holding the cannula 100 steady, or by applying a retraction force on the cannula 100 as a pushing, or advancing force is placed on the capsule 212. For reference, the end of the catheter 202 including the pusher 218 is hidden from view by the cannula 100 in FIG. 6, but has been advanced far enough in FIG. 7 to be visible. The first portion 532 of the anchor structure 102 may be pushed against the first side of the tissue wall 530 by advancing the cannula 100.

As shown in FIG. 7, the pusher 218 may be coupled to the catheter 202 or otherwise form an extension of the catheter 202 (e.g., integrally formed to a body of the catheter 202). The pusher 218 is arranged to couple the capsule 212 to the catheter 202 so that the capsule 212 may be advanced relative to the cannula 100 and subsequently withdrawn in a collapsed state through the cannula 100 from the body of a patient. As shown in FIG. 7, the capsule 212 may also facilitate deployment of a second portion 534 of the anchor structure 102 against a second side of the tissue wall 530. The delivery catheter 202 may be advanced relative to the cannula 100 to deploy the second portion 534 of the anchor structure 102 by forcing the capsule 212 from the cannula 100 by applying a force to the delivery catheter 202 to pushing the capsule 212 beyond the second portion 534 of the anchor structure 102. As shown in FIG. 7, the capsule 212 is extended beyond the second portion 534 of the anchor structure 102 after the anchor structure 102 is arranged with the tissue wall 530. This configuration of the anchor structure may define the second, deployed configuration.

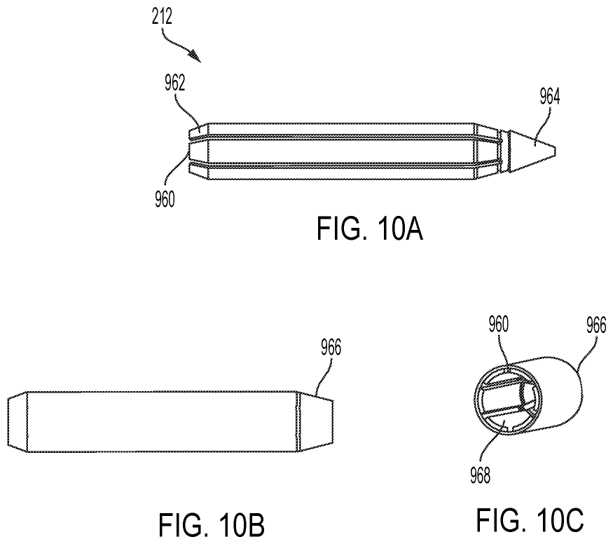

The capsule 212, as shown in FIG. 8, may be configured to collapse and be withdrawn through the cannula 100 after deployment of the anchor structure 102 as described in further detail below with respect to FIG. 10. The capsule 212 may be withdrawn through the cannula 100 from the second deployed configuration to the first delivery configuration by withdrawing the catheter 202, including the pusher 218 (FIG. 7) coupled to the capsule 212. The cannula 100 may be purged using the hemostasis valve 204. In instances where the cannula 100 is used for cardiac assistance or other flow control/enhancement, the cannula 100 may be coupled to a pump. The cannula 100 may be further held in place by using a suture sleeve 206 arranged about the cannula 100 to secure an opposite end of the cannula 100 in the patient.

FIGS. 10A-C provide an illustration of an example capsule 212 and associated features for the delivery system 200, shown in FIGS. 3-9, in accordance with various aspects of the present disclosure. The capsule 212, in certain instances, may be segmented to facilitate collapse and withdrawal through the cannula 100 to the first, delivery configuration after deployment of the anchor structure 102 as described in detail above. The capsule 212, as shown, may be discontinuous about a circumference to facilitate collapse of the capsule 212 in response to tension applied to the delivery catheter 202 as discussed in detail above.

In certain instances, the capsule 212 may include a series of segments 960 extending along at least a portion of a length of the capsule 212 and spaced about the circumference of capsule 212. In certain instances, the series of segments 960 may be gaps in the capsule 212 that extend between an outer surface and an inner surface of the capsule 212, and in other instances, the series of segments 960 may include breaks that do not form a gap in the capsule 212. In certain instances, the series of segments 960 may be continuous along the capsule 212 from beginning to end, and in other instances, the series of segments 960 may be continuous along only a portion of the capsule 212.

In certain instances, the capsule 212 includes a tapered proximal end 962 and a tapered distal end 964 to facilitate collapse and withdrawal through the cannula 100 after deployment of the anchor structure 102. In certain instances, the tapered distal end 964 includes a longer length than the tapered proximal end 962. In addition, the tapered distal end 964 may taper at the same angle or at a different (e.g., higher or lower) angle than the tapered proximal end 962. In certain instances, one or both of the tapered proximal end 962 and the tapered distal end 964 may include the series of segments 960. In other instances, neither of the tapered proximal end 962 and the tapered distal end 964 may include the series of segments 960. As shown, the tapered proximal end 962 includes portions of the series of segments 960 and the tapered distal end 964 includes a continuous outer circumference.

In certain instances, the tapered proximal end 962 is configured to facilitate collapse of the capsule 212 within the cannula 100 in response to tension applied to the delivery catheter 202. The tapered proximal end 962 may draw or ease the capsule 212 into the cannula 100 or delivery catheter 202 (which may include an elastic tube 966 that holds and collapses the capsule 212 as shown in FIG. 10B). The elastic tube may be configured to constrain the capsule 212 and may form a portion of the delivery catheter 202. In certain instances, the capsule 212 further includes a hydrophilic coating on the capsule 212 configured to facilitate advancement of the capsule 212 through the tissue wall, for example. In certain instances, the capsule 212 further includes additives or discrete elements visible under fluoroscopy to facilitate visualization during delivery.

As shown in FIG. 10C, the capsule 212 includes an inner surface 968 that facilitates constraining of the anchor structure 102 discussed above. The anchor structure 102 may ride or slide along the inner surface 968 of the capsule 212.

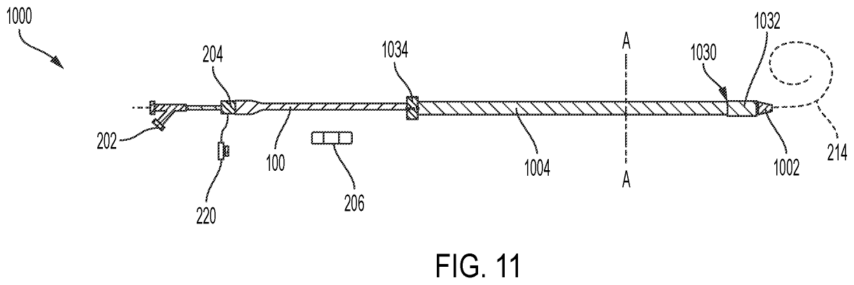

FIG. 11 is an illustration of another example delivery system 1000 for a cannula 100, in accordance with various aspects of the present disclosure. The delivery system 1000 may facilitate delivery of the cannula 100 to a target location within the patient. The delivery system 1000 may be used to place the cannula 100 and an anchor structure 102 (as described in detail with reference to FIGS. 3-9) coupled or attached to an end of the cannula 100 at the target location. The system 1000 includes a balloon 1002 in place of the capsule 112 described above and a delivery sheath 1004.

The delivery sheath 1004 may be between about 24 French and 30 French and may extend from an entry point to a target location for anchoring of the cannula 100. In certain instances, the delivery system 1000 may be used to cross or open a space between compartments of the patient or within tissue of the patient to place the cannula 100 and the anchor structure 102. In certain instances, the delivery system 1000 also includes a hemostasis plug 204 as described in detail above.

The delivery system 1000 also includes a delivery catheter 202 that is configured to extend through the cannula 100 with the balloon 1002 arranged at an end of the delivery catheter 202. In certain instances, a guidewire 214 may be used to direct the delivery system 200 to the target location.

The delivery sheath 1004 may be advanced across the target location to deploy the anchor structure 102. Rather than the capsule 112 containing the anchor structure 102, the delivery sheath 1004 may contain the anchor structure 102. In certain instances, a distal end portion 1030 of the delivery sheath 1004 may have a radial strength that allows for constraining and containment of the anchor structure 102. In certain instances, the distal end portion 1030 is configured to constrain the anchor structure 102 in the delivery configuration and deploy the anchor structure 102 in response to a force applied to the delivery sheath 1004 to move the distal end portion 1030 relative to the cannula 100. The distal end portion 1030 may be operable to be peeled or torn from about the cannula 100 in some embodiments. In certain instances, the delivery sheath 1004 may be formed of or comprise expanded polytetrafluoroethylene (ePTFE). The distal end portion 1030 of the delivery sheath 1004 may be densified, include stent elements, or otherwise be stiffened to enhance the radial strength of a portion of the delivery sheath 1004 while one or more remaining portions of the delivery sheath 1004 remain relatively more flexible to help maintain desired flexibility. As a result, the distal end portion 1030 of the delivery sheath 1004 may include a greater hoop strength than other portions of the delivery sheath 1004.

The delivery sheath 1004 may include a hub 1034 at an opposite proximal end to the distal end portion 1030 of the delivery sheath 1004. The hub 1034 may be configured to facilitate entry of the cannula 100 into the patient at the entry point. The delivery sheath 1004 and the cannula 100 may be introduced into the patient concurrently, or as part of a single step. In other examples, the cannula 100 is introduced into the delivery sheath 1004 using the catheter 202 after the delivery sheath 1004 has been placed in the vasculature and directed to the target site. In certain instances, the hub 1034 may be splittable to remove the delivery sheath 1004 from about the cannula 100. The hub 1034 may facilitate splitting of the delivery sheath 1004 to remove the delivery sheath 1004 from about the cannula 100. In some instances, the hub 1034 is configured to be slide over the end of the proximal end of the cannula 100 (e.g., and hemostasis plug 204) to remove the delivery sheath 1004 from the cannula 100 after delivery and deployment of the cannula 100 at its intended location. The hub 1034 may also be flareable, expandable, or otherwise enlargeable to facilitate removal of the hub 1034, and the delivery sheath 1004, from the cannula 100.

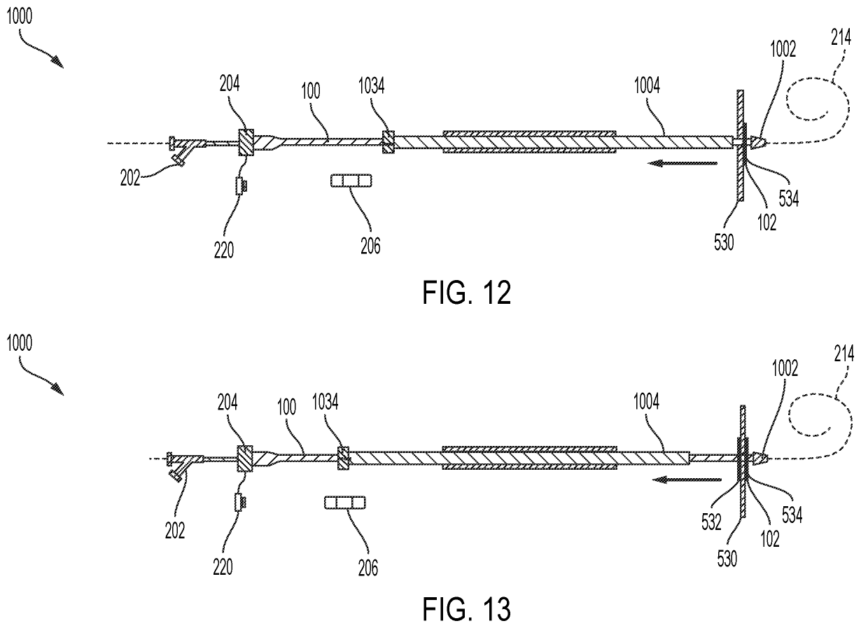

As shown in FIG. 12, after the delivery catheter 202 (and optionally the delivery sheath 1004) has forced the balloon 1002 through an existing opening and/or dilated the opening. The delivery sheath 1004 may be concurrently or separately advanced across the opening at target location 530. The target location 530 may be a tissue wall 530, which may include a heart wall, septum, a vessel wall, or other tissue wall, for example. After the delivery sheath 1004 is advanced through an opening in the tissue wall 530, the delivery sheath 1004 may be withdrawn to deploy a second (distal) portion 534 of the anchor structure 102 on a second side of the tissue wall 530 in response to a force (e.g., advancing force) applied to the delivery catheter 202 and/or a force applied to the delivery sheath 1004 (e.g., retracting force). The force may be applied by a physician. The second (distal) portion 534 of the anchor structure 102 may be retracted against the second side of the tissue wall 530 is shown in FIG. 12 by retracting the cannula 100. In certain instances, the delivery catheter 202 is a balloon catheter and is configured to extend through the hub 1034 and dilate the target location 530 for the cannula 100 and anchor structure 102. The balloon 1002 may remain inflated to stabilize the cannula 100 during deployment of the anchor structure 102.

As shown in FIG. 13, the delivery sheath 1004 may be further withdrawn to expose a first (proximal) portion 532 of the anchor structure 102 against a first side of the tissue wall 530. The delivery catheter 202 and the balloon 1002 may be withdrawn from the body through the cannula 100 after deployment of the anchor structure 102. A user may apply a force to split the hub 1034 and further split the delivery sheath 1004 from about the cannula 100. In other examples, the hub 1034 may be flared, re-sizeable (e.g., expandable) or pre-sized to be retracted from over the cannula 100.

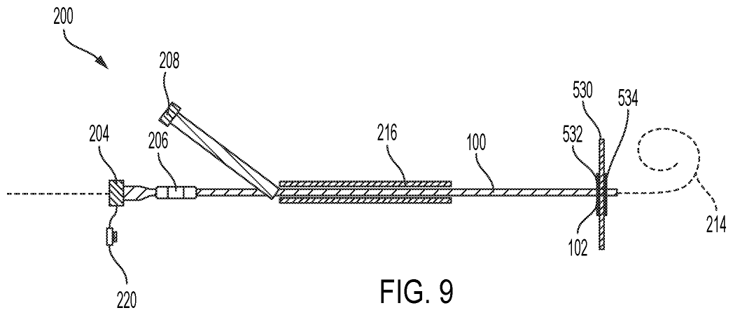

The cannula 100 may be further held in place by using a suture sleeve 206 arranged about the cannula 100 to secure an opposite end of the cannula 100 to the patient (e.g., in a similar position to that shown in FIG. 9).

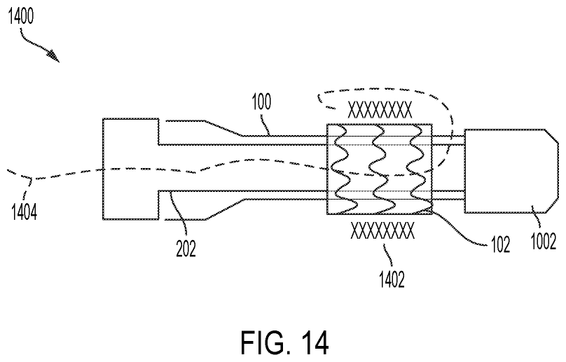

FIG. 14 is an illustration of another example delivery system 1400 for a cannula 100, in accordance with various aspects of the present disclosure. The delivery system 1400 may be directed to a target location as described in detail above for delivery of the cannula 100 and anchor structure 102 to the target location. The delivery system 1400 may include a delivery catheter 202 with a dilator (e.g., a balloon 1002).

The delivery system 1400 may also include a zipper constraint 1402 that is integrated with the delivery catheter 202. In certain instances, and as is shown, the delivery catheter 202 and balloon 1002 may be arranged within the cannula 100 and provide column strength for cannula 100 trackability while also serving as an inflatable lead to reduce trauma to vasculature tissue. The zipper constraint 1402 may be configured constrain the anchor structure 102 to the cannula 100 to reduce a profile during delivery of the cannula 100 to the target location 530. In addition, the zipper constraint 1402 may also minimize the step from the zipper constraint 1402 to the balloon 1002. Further, the zipper constraint 1402 may allow for delivery of the cannula 100 and anchor structure 102 without the use of a sheath.

As shown in FIG. 14, the zipper constraint 1402 may be configured to constrain the anchor structure 102 while leaving the remaining length of the cannula 100 uncovered by the zipper constraint 1402. The zipper constraint 1402 may be configured to constrain the anchor structure 102 against the cannula 100 in the delivery configuration and release to deploy the anchor structure 102 in response to a force applied by a user to a deployment line 1404 coupled to the zipper constraint 1402. This zipper constraint 1402 may be configured to reduce the amount of slack of the deployment line 1404 a user will need to pull to achieve complete anchor structure 102 deployment. Tension may be applied to the deployment line 1404 to deploy the zipper constraint 1402 and release the anchor structure 102 at a target location. The zipper constraint 1402 may deploy in stages to release one portion of the anchor structure 102 on one side of a tissue wall prior to deploying another potion of the anchor structure 102 on an opposite side of the tissue wall. For further discussion of the zipper constraint 1402, which may be an interwoven or knit series of fibers, reference may be made to U.S. Pat. No. 6,315,792 (“Armstrong et al.”) and U.S. Pat. No. 8,308,789 (“Armstrong”) which provide suitable examples of such constraint features.

A biocompatible material for the graft components, discussed herein, may be used. In certain instances, the graft components may include a fluoropolymer, such as a polytetrafluoroethylene (PTFE) polymer or an expanded polytetrafluoroethylene (ePTFE) polymer. In some instances, the graft components may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

In addition, nitinol (NiTi) may be used as the material of the frame of the anchor structure (and any of the anchor elements discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

In certain instances, the coating, as discussed in detail above, may include bio-active agents in addition to heparin or alternatively to heparin. The agents can include, but are not limited to, vasodilator, anti-coagulants, anti-platelet, anti-thrombogenic agents.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A delivery system comprising:
a cannula having an anchor structure configured from transition from a first, delivery configuration to a second, deployed configuration;
a delivery sheath;
a capsule receiving the anchor structure in the first, delivery configuration; and
a delivery catheter including a pusher configured to advance the capsule relative to the cannula, the capsule being arranged at an end of the delivery catheter, the delivery catheter being configured to extend through the cannula and operable to advance the capsule through an opening, deploy a first portion of the anchor structure on a first side of the opening from the capsule, and deploy a second portion of the anchor structure against a second side of the opening such that the anchor structure defines the second, deployed configuration, and optionally collapse the capsule to be withdrawn through the cannula from the second, deployed configuration to the first, delivery configuration after deployment of the anchor structure in response to force applied to the delivery catheter by a physician.

2. The delivery system of claim 1, wherein the capsule is segmented to facilitate collapse and withdrawal through the cannula to the first, delivery configuration after deployment of the anchor structure.

3. The delivery system of claim 2, wherein the capsule is discontinuous about a circumference of the capsule to facilitate collapse of the capsule in response to tension applied to the delivery catheter.

4. The delivery system of claim 3, wherein the capsule includes a series of segments extending along a length and spaced about the circumference of the capsule.

5. The delivery system of claim 4, wherein the capsule includes a tapered proximal end and a tapered distal end to facilitate collapse and withdrawal through the cannula after deployment of the anchor structure.

6. The delivery system of claim 5, wherein the tapered distal end of the capsule includes a continuous outer circumference and the tapered proximal end includes portions of the series of segments.

7. The delivery system of claim 6, wherein the tapered proximal end is configured to facilitate collapse of the capsule within the cannula in response to tension applied to the delivery catheter to draw the capsule into the cannula.

8. The delivery system of claim 7, further comprising an elastic tube configured to constrain the capsule.

9. The delivery system of claim 1, wherein the capsule includes a hydrophilic coating configured to facilitate visualization of the capsule and advancement of the capsule through the opening.

10. The delivery system of claim 1, further comprising a hemostasis plug.

11. A delivery system for a cannula including an anchor structure, the delivery system comprising:
a hub configured to facilitate entry of the cannula into a patient at an entry point;
a balloon catheter configured to extend through the hub and dilate a target location for the cannula and the anchor structure; and
a delivery sheath configured to extend between the entry point and the target location, the delivery sheath including:
a distal end portion configured to constrain the anchor structure in a delivery configuration and deploy the anchor structure in response to a force applied to the delivery sheath to move the distal end portion relative to the cannula.

12. The delivery system of claim 11, wherein the distal end portion is operable to be peeled or torn from about the cannula.

13. The delivery system of claim 11, wherein the hub is configured to facilitate splitting of the delivery sheath to remove the delivery sheath from about the cannula.

14. The delivery system of claim 11, wherein the balloon catheter is configured to hold the cannula at the target location while the delivery sheath is withdrawn to deploy the anchor structure.

15. The delivery system of claim 11, wherein the delivery sheath comprises expanded polytetrafluoroethylene (ePTFE).

16. The delivery system of claim 11, wherein the distal end portion includes a greater hoop strength than an adjacent portion of the delivery sheath.

17. A delivery system comprising:
a cannula having an anchor structure arranged with the cannula;
a delivery catheter including a balloon configured dilate a target location for the cannula and anchor structure; and
a zipper constraint configured to constrain the anchor structure against the cannula in a delivery configuration and release to deploy the anchor structure in response to a force applied to a deployment line coupled to the zipper constraint by a user.

18. The delivery system of claim 17, wherein the delivery catheter and the balloon are configured to provide column strength to the cannula for trackability while also serving as an inflatable lead to reduce trauma to vasculature tissue.

19. The delivery system of claim 18, wherein the zipper constraint is configured constrain the anchor structure to the cannula to reduce a profile of the delivery system during delivery of the cannula to a target location.
